(19) United States
(12) Patent Application Publication
(10) Pub. No.: US 2016/0083409 A1
(43) Pub. Date: Mar. 24, 2016

(54) METHOD FOR THE PREPARATION OF alpha-SUBSTITUTED ACRYL ALDEHYDES

(71) Applicant: Council of Scientific and Industrial Research, New Delhi (IN)

(72) Inventors: Ramana Venkata Chepuri, Pune (IN); Chandrababu Naidu Kona, Pune (IN)

(21) Appl. No.: 14/578,390

(22) Filed: Dec. 20, 2014

(30) Foreign Application Priority Data
Sep. 24, 2014 (IN) 2735/DEL/2014

(51) Int. Cl. C07F 15/02; C07D 209/12; C07C 49/217; C07D 207/333; C07C 49/255; C07C 225/22

(52) U.S. Cl. CPC C07F 15/02; C07C 49/255; C07C 225/22; C07C 49/217; C07D 207/333; C07D 209/12

(57) ABSTRACT

The invention discloses simple and rapid method for the preparation of α-substituted acryl aldehydes. More particularly, the invention discloses a method for the preparation of α-substituted acryl aldehydes via a gold-catalysed [1,3] rearrangement of the allenyl ethers with a record turnover frequency of 4600 h⁻¹ at 0.05 mol % of the catalyst concentration in homogeneous gold(I) catalysis. The α-substituted acryl aldehydes synthesized by the instant process are used as building blocks in organic synthesis.

### CLAIM OF PRIORITY

This application claims the benefit of priority of India Patent Application No. 2735/DEL/2014, filed on Sep. 24, 2014, the benefit of priority of which is claimed hereby, and which is incorporated by reference herein in its entirety.

### FIELD OF INVENTION

The invention relates to a simple and rapid method for the preparation of α-substituted acryl aldehydes. More particularly, the invention relates to a method for the preparation of α-substituted acryl aldehydes via a gold-catalysed [1,3] rearrangement of the allenyl ethers with a record turnover frequency of 4600 h⁻¹ at 0.05 mol % of the catalyst concentration in homogeneous gold(I) catalysis. The α-substituted acryl aldehydes synthesized by the instant process are used as building blocks in organic synthesis.

### BACKGROUND AND PRIOR ART

During the last decade, gold-complexes enabled organic synthesis with a remarkable reactivity and with an ability to catalyse diverse organic transformations. In particular, the selective activation of allenes by gold-complexes, followed by the subsequent inter and intramolecular nucleophilic additions and [3,3]-sigmatropic rearrangements (such as Claisen and Cope rearrangements), has received substantial attention. Surprisingly, the utilization of allene units and the gold catalysts in the [1,3] O-C rearrangement has been less explored by the research community. The [1,3] rearrangement reaction of vinyl ethers in presence of Lewis acids constitutes an important C—C bond forming reaction that has attracted considerable attention over the last two decades. Recently, the complexes of Pd, Co, Ir and Ru have been shown to be effective for this purpose, refer [Pd]: a) B. M. Trost and J. Xie, J. Am. Chem. Soc., 2006, 128, 6044; b) D. M. D'Souza, F. Rominger and T. J. J. Midler, Chem. Commun, 2006, 4096; c) S. Zhu, L. Wu and X. Huang, RSC Adv., 2012, 2, 132; [Co]: d) S. J. Meek, F. Pradaux, D. R. Carbery, E. H. Demont and J. P. A. Harrity, J. Org. Chem., 2005, 70, 10046; [Ir]: e) H-Y. Wang, D. S. Mueller, R. M. Sachwani, R. Kapadia, H. N. Londino and L. L. Anderson, J. Org. Chem., 2011, 76, 3203; [Ru]: f) N-a. Harada, T. Nishikata and H. Nagashima, Tetrahedron, 2012, 68, 3243. The [1,3] rearrangement reactions involving the Lewis acid catalysts are generally postulated to proceed through the heterolytic cleavage of the O—R bond of the vinyl ether and via the formation of an intermediate ion-pair comprising the carbocationic species R+ and an enolate counterpart and the success of this reaction depends upon a careful choice of Lewis acids, as well as the selection of appropriate R groups that can stabilize the transient carbocation. Considering the prerequisite of ‘an ion-pair mechanism’ for the success of a “[1,3] rearrangement” and the formation of ion pairs with the catalytically active cations in the Au[I]-catalyzed reactions, the instant inventors envisioned that the [1,3] rearrangement of the allenyl ethers would constitute a general protocol for the synthesis of C2-substituted acryl aldehyde derivatives. Further, there is ample literature available on gold-catalysis. The formation of trace amounts of [1,3] rearrangement products has been noticed during [3,3] Claisen rearrangement of propargyl vinyl ethers and allyl vinyl ethers by Toste and Krafft, refer B. D. Sherry and F. D. Toste, J. Am. Chem. Soc., 2004, 126, 15978; Y. Liu, J. Qian, S. LouandZ. Xu, Synlett, 2009, 2971; J. R. Vyvyan, H. E. Dimmitt, J. K. Griffith, L. D. Steffens and R. A. Swanson, Tetrahedron Lett., 2010, 51, 6666 and M. E. Krafft, K. M. Hallal, D. V. Vidhani and J. W. Cran, Org. Biomol. Chem., 2011, 9, 7535-7538.

On the other hand, in the case of the reactions involving allenyl ethers and gold-complexes, Cui and co-workers have recently reported the gold-catalysed nucleophilic addition of alcohols at the C1 of allenyl(p-methoxybenzyl) ether (1c) as shown in scheme I, refer D-M. Cui, Z-L. Zhengand C. Zhang, J. Org. Chem., 2009, 74, 1426.

The α-substituted acryl aldehydes are used as building blocks in organic synthesis. However, there is no efficient synthesis for the preparation of the same available in the art. In the light of the foregoing, there is a need in the art to provide an efficient method for the preparation of α-substituted acryl aldehydes via a gold-catalysed [1,3] O—C rearrangement of the allenyl ethers.

### OBJECT OF INVENTION

Accordingly, the objective of the present invention is to provide an efficient method for the preparation of α-substituted acryl aldehydes via a gold-catalysed [1,3] O—C rearrangement of the allenyl ethers, for which protection is sought.

### SUMMARY OF THE INVENTION

In line with the above objectives, the invention discloses a process for the preparation of α-substituted acryl aldehydes of Formula 2,

wherein n represents 0, 1
“A” is selected from the group consisting of phenyl; benzyl; substituted or unsubstituted furyl, thiophenyl, pyrrolyl, pyridyl, benzofuryl, benzothiophenyl, indolyl, wherein substituents are selected from N—(C₁-C₄)alkyl or, N-arylalkyl;
R is selected from the group consisting of hydrogen, aryl, arylalkyl, (C₁-C₆) linear or branched alkyl;
R¹, R² and R³ are similar or different and independently selected from the group consisting of hydrogen, (C₁-C₄)alkoxy, N—(R⁴)₂, where R⁴ is (C₁-C₆)alkyl; with a proviso when n is 0, then “A” is selected from the group consisting of benzocyclohexyl and ferrocenyl;
comprising subjecting allenyl ethers of Formula 1 to Au(I) complex catalysed [1, 3] O→C rearrangement to obtain α-substituted acryl aldehydes of Formula 2 with turnover frequency of 4600 h⁻¹.

The Au(I) complex according to the invention is selected from the group consisting of AuCl₃; AuBr₃, AuCl(PPh₃); AuCl(PMe₃); AuCl(Biphenyl(tBu)₂), preferably the Au(I) complex is AuCl(PPh₃).

The Au(I) complex according to the process of the invention is used in an amount of 0.05 mol % in homogeneous catalysis.

The process according to the invention additionally comprise a silver salt to facilitate the [1, 3] O→C rearrangement.

The silver salt is selected from the group consisting of AgSbF₆; AgOAc; AgOTf and AgNTf₂, the silver salt preferably is AgSbF₆.

The process according to the invention, wherein, the molar ratio of Au [I] complex and additive is in the range of 1:6 to 1:3, more preferably the molar ratio is 1:3.

In a preferred aspect, the invention provides a process for the preparation of α-substituted acryl aldehydes of Formula 2, which comprises reacting allenyl ethers of Formula 1 with Au(I) complex in presence of aprotic solvents at a temperature of 0° C. to room temperature, to obtain α-substituted acryl aldehydes of Formula 2.

The present invention further encompasses compounds of Formula 2

wherein n represents 0,1
“A” is selected from the group consisting of phenyl; benzyl; substituted or unsubstituted furyl, thiophenyl, pyrrolyl, pyridyl, benzofuryl, benzothiophenyl, indolyl, wherein substituents are selected from N—(C₁-C₄)alkyl or, N-arylalkyl;
R is selected from the group consisting of hydrogen, aryl, arylalkyl, (C₁-C₆) linear or branched alkyl;
R¹, R² and R³ are similar or different and independently selected from the group consisting of hydrogen, (C₁-C₄)alkoxy, N—(R⁴)₂, where R⁴ is (C₁-C₆)alkyl; with a proviso when n is 0, then “A” is selected from the group consisting of benzocyclohexyl and ferrocenyl.

In a more preferred aspect, the representative compounds of Formula 2 are as given below.
(i) 2-(4-Methoxybenzyl)acrylaldehyde;
(ii) 2-((4-Methoxyphenyl)(phenyl)methyl)acrylaldehyde;
(iii) 3-(4-Methoxyphenyl)-2-methylene-4-phenylbutanal;
(iv) 3-(4-Methoxyphenyl)-2-methyleneheptanal′
(v) 2-(2,4-Dimethoxybenzyl)acrylaldehyde;
(vi) 2-((2,4-Dimethoxyphenyl)(phenyl)methyl)acrylaldehyde;
(vii) 2-(3,4-Dimethoxybenzyl)acrylaldehyde;
(viii) 2-((3,4-Dimethoxyphenyl)(phenyl)methyl)acrylaldehyde;
(ix) 3-(3,4-Dimethoxyphenyl)-2-methylene-4-phenylbutanal;
(x) 2-Methylene-4-phenyl-3-(3,4,5-trimethoxyphenyl)butanal;
(xi) 2-(Phenyl(3,4,5-trimethoxyphenyl)methyl)acrylaldehyde;
(xii) 2-(4-(Dimethylamino)benzyl)acrylaldehyde;
(xiii) 2-((4-(Dimethylamino)phenyl)(phenyl)methyl)acrylaldehyde;
(xiv) 3-(4-(Dimethylamino)phenyl)-2-methyleneheptanal;
(xv) 2-((3,5-Dimethoxyphenyl)(phenyl)methyl)acrylaldehyde;
(xvi) 2-Benzhydrylacrylaldehyde;
(xvii) 2-(6-Methoxy-1,2,3,4-tetrahydronaphthalen-1-yl)acrylaldehyde′
(xviii) 2-((4-Methoxyphenyl)(phenyl)methyl)-3-phenylacrylaldehyde;
(xix) 2-((2,4-Bimethoxyphenyl)(phenyl)methyl)-3-phenylacrylaldehyde;
(xx) Cyclopenta-2,4-dien-1-yl(2-(2-formylallyl)cyclopenta-2,4-dien-1-yl)iron;
(xxi) 3-(1-Methyl-1H-pyrrol-2-4)-2-methylenebutanal;
(xxii) 2-Methylene-3-(thiophen-2-yl)butanal;
(xxiii) 3-(1-Benzyl-1H-pyrrol-2-4)-2-methyleneheptanal;
(xxiv) 3-(Benzofuran-2-yl)-2-methylenebutanal;
(xxv) 3-(Benzo[b]thiophen-2-yl)-2-methylenebutanal;
(xxvi) 3-(1-Benzyl-1H-indol-3-yl)-2-methyleneheptanal;

### DETAILED DESCRIPTION

The invention will now be described in detail in connection with certain preferred and optional embodiments, so that various aspects thereof may be more fully understood and appreciated.

Accordingly, the invention discloses a process for the preparation of α-substituted acryl aldehydes of Formula 2,

wherein n represents 0,1
“A” is selected from the group consisting of phenyl; benzyl; substituted or unsubstituted furyl, thiophenyl, pyrrolyl, pyridyl, benzofuryl, benzothiophenyl, indolyl, wherein substituents are selected from N—(C₁-C₄)alkyl or N-arylalkyl;
R is selected from the group consisting of hydrogen, aryl, arylalkyl, (C₁-C₆) linear or branched alkyl;
R¹, R² and R³ are similar or different and independently selected from the group consisting of hydrogen, (C₁-C₄)alkoxy, N—(R⁴)₂, where R⁴ is (C₁-C₆)alkyl; with a proviso when n is 0, then “A” is selected from the group consisting of benzocyclohexyl and ferrocenyl;
comprising subjecting allenyl ethers of Formula 1 to Au(I) complex catalysed [1, 3] O→C rearrangement to obtain α-substituted acryl aldehydes of Formula 2 with turnover frequency of 4600 h⁻¹.

The Au(I) complex according to the invention is selected from the group consisting of AuCl₃; AuBr₃, AuCl(PPh₃); AuCl(PMe₃); AuCl(Biphenyl(tBu)₂), preferably the Au(I) complex is AuCl(PPh₃).

The Au(I) complex according to the process of the invention is used in an amount of 0.05 mol % in homogeneous catalysis.

According to an embodiment, a silver salt is optionally used as an additive to facilitate the [1,3] O-C rearrangement. The silver salt is selected from the group consisting of AgSbF₆; AgOAc; AgOTf and AgNTf₂, the silver salt preferably AgSbF₆.

In a preferred embodiment, the molar ratio of Au [I] complex and the additive is in the range of 1:6 to 1:3, more preferably the molar ratio is 1:3.

Accordingly, in a preferred embodiment, the process comprises reacting allenyl ethers of Formula 1 with Au(I) complex and silver salt in a molar ratio of 1:3, in presence of aprotic solvents at a temperature of 0° C. to room temperature, to obtain α-substituted acryl aldehydes of Formula 2.

In a typical embodiment, allenyl(p-methoxybenzyl) ether 1c has been subjected to [Au]-catalysed [1,3] O→C rearrangement by considering the fact that electron-donating groups on the aryl ring will stabilize the intermediate benzylcationformed. [Au]-catalysed [1,3] O→C rearrangement of the invention is carried out in aprotic solvents to facilitate the reaction in the requisite direction (Scheme II).

In another embodiment, the invention demonstrates the substrate scope of the reaction by selecting various allenyl ethers 1a-1c, having, respectively, the decyl, benzyl and p-methoxybenzyl units as R groups to verify the limitations inter alia to learn about how the stability of the in situ generated carbocation will influence the outcome of the [1,3] O→C rearrangement.

In a further embodiment, Tte exploratory experiments were carried out employing 2 mol % of catalyst in dichloromethane as the solvent with different Au salts to assess the suitability of the cation. While the reactions with Au [III] salts ended up with the hydrolysis of the allenyl ethers 1a-1c, however, in the case of the Au[I]-complexes, when employed alone, the starting materials were recovered intact.

According to the invention, the Au(I) complexes are selected from the group consisting of AuCl₃, AuBr₃, AuCl(PPh₃); AuCl(PMe₃); AuCl(Biphenyl(^{t}Bu)₂). A preferable Au(I) complex is AuCl(PPh₃).

In a further embodiment, an additive, preferably a silver salt is added to enhance the rate of reaction. The silver salts according to the invention are selected from the group consisting of AgSbF₆; AgOAc; AgOTf and AgNTf₂. One preferable silver salt is AgSbF₆.

According to the invention, the combination of the Au(I)-complexes with the additive AgSbF₆ resulted in the quantitative conversion of 1c within 5 minutes at 0° C. in dichloromethane and 2-(4-methoxybenzyl)acrylaldehyde (2c) was obtained in excellent yield. Under similar conditions, the allenyl ethers 1a and 1b hydrolysed immediately after the addition of the catalyst. Changing either the ligand on the Au[I]-complex or the counter anion did not provide any promising results with the substrates 1a and 1b. Controlled experiments revealed that, with the silver salts [5-10 mol %] AgOAc, AgOTf and AgNTf₂, only the hydrolysis of the allenyl ether 1c was observed. With AgSbF₆, the reaction was sluggish and the acryl aldehyde 2c was obtained in moderate yields (table 1). These experiments clearly demonstrate that the active catalyst involved in the [1,3] rearrangement was the in situ generated cationic [Au]-complex and that the weakly coordinating counter anion favours the rearrangement.

```
TABLE 1
  1a (R = nC₉H₁₇) 1b (R = Ph) 1c (R = p-MeOC₆H₄)
  2
```

| Entry | Substrate | Catalyst | additive | Yield |
|---|---|---|---|---|
| 1 | 1a | AuCl₃ | — | Hydrolysis |
| 2 | 1b | AuCl₃ | — | Hydrolysis |
| 3 | 1c | AuCl₃ | — | Hydrolysis |
| 4 | 1a | AuBr₃ | — | Hydrolysis |
| 5 | 1b | AuBr₃ | — | Hydrolysis |
| 6 | 1c | AuBr₃ | — | Hydrolysis |
| 7 | 1a | AuCl(PPh₃) | — | No reaction |
| 8 | 1b | AuCl(PPh₃ | — | No reaction |
| 9 | 1c | AuCl(PPh₃ | — | No reaction |
| 10 | 1a | AuCl(PPh₃) | AgSbF₆ | Hydrolysis |
| 11 | 1b | AuCl(PPh₃) | AgSbF₆ | Hydrolysis |
| 12 | 1c | AuCl(PPh₃) | AgSbF₆ | 70-96% |
| 13 | 1a | AgSbF₆ | — | Hydrolysis |
| 14 | 1b | AgSbF₆ | — | Hydrolysis |
| 15 | 1c | AgSbF₆ | — | 27% |
| 16 | 1a | AgOTf | — | No reaction |
| 17 | 1b | AgOTf | — | No reaction |
| 18 | 1c | AgOTf | — | No reaction |
| 19 | 1a | AgNTf₂ | — | Hydrolysis |
| 20 | 1b | AgNTf₂ | — | Hydrolysis |
| 21 | 1c | AgNTf₂ | — | Hydrolysis |
| 22 | 1c | AgOAc | — | No reaction |
| 23 | 1a | AuCl₃ | AgSbF₆ | Hydrolysis |
| 24 | 1b | AuCl₃ | AgSbF₆ | Hydrolysis |
| 25 | 1c | AuCl₃ | AgSbF₆ | Hydrolysis |
| 26 | 1a | AuCl(PMe₃) | AgSbF₆ | Hydrolysis |
| 27 | 1b | AuCl(PMe₃) | AgSbF₆ | Hydrolysis |
| 28 | 1c | AuCl(PMe₃) | AgSbF₆ | 45-55% |
| 29 | 1a | AuCl(PMe₃) | AgOTf | Hydrolysis |
| 30 | 1b | AuCl(PMe₃) | AgOTf | Hydrolysis |
| 31 | 1c | AuCl(PMe₃) | AgOTf | Hydrolysis |
| 32 | 1a | AuCl(PPh₃) | AgNTf₂ | Hydrolysis |
| 33 | 1b | AuCl(PPh₃) | AgNTf₂ | Hydrolysis |
| 34 | 1c | AuCl(PPh₃) | AgNTf₂ | Hydrolysis |
| 35 | 1a | AuCl(PMe₃) | AgNTf₂ | Hydrolysis |
| 36 | 1b | AuCl(PMe₃) | AgNTf₂ | Hydrolysis |
| 37 | 1c | AuCl(PMe₃) | AgNTf₂ | Hydrolysis |
| 38 | 1a | AuCl(Biphenyl)(^{t}Bu)₂) | AgSbF₆ | Hydrolysis |
| 39 | 1b | AuCl(Biphenyl)(^{t}Bu)₂) | AgSbF₆ | Hydrolysis |
| 40 | 1c | AuCl(Biphenyl)(^{t}Bu)₂) | AgSbF₆ | 40-45% |

As the reaction with 2 mol % of the catalyst was found to be almost instantaneous, further experiments were carried out for the optimal concentration of the catalyst required at ambient temperature. Accordingly, controlled experiments were conducted with the allenyl ether 1c at different concentrations of the catalyst, varied from 0.0125-0.05 mol %, by keeping the catalyst to additive ratio as 1:3. Out of all the concentrations, the reaction with 0.05 mol % catalyst at 0° C. (5 min duration, S/C=2800) was found to be optimal for C—C bond formation and gave the required rearranged product 2c in 97% yield (on 1 g scale) with the highest TOF (4600 h⁻¹). For lower concentrations like 0.0125 mol %, the reaction was sluggish at room temperature and, when refluxed, the reaction proceeded within 12 h (80% conversion), after which there was no further conversion of 1c, providing 2c in 90% isolated yield (S/C=9072), as depicted in table 2.

Optimization studies for the best turnover frequency

TABLE 2

| S. No | X (mol %) | temp | time | | Yield^{a} | S/C | TOF(h⁻¹) |
|---|---|---|---|---|---|---|---|
| 1 | 0.050 | 0° C. | 05 | min | 96% | 2800 | 4600 |
| 2 | 0.045 | 0° C. | 30 | min | 92% | 3100 | 4088 |
| 3 | 0.040 | 0° C. | 60 | min | 89% | 3500 | 2225 |
| 4 | 0.035 | 25° C. | 08 | h | 87% | 4000 | 310 |
| 5 | 0.030 | 25° C. | 10 | h | 86% | 4600 | 286 |
| 6 | 0.025 | 25° C. | 16 | h | 86% | 5600 | 215 |
| 7 | 0.0125 | reflux | 12 | h | 90%^{b} | 9072 | 600 |

3)S/C = Substrate/Catalyst Concentration
^{a}isolated yields;
^{b}based on the 20% starting material recovered

In yet another embodiment, the substrate scope of the current invention is represented in table 3.

TABLE 3
Scope of [Au]-catalyzed [1.3] rearrangement reaction

| | | | | |
|---|---|---|---|---|
| 2c, R = R′ = H (91%)* 2d, R = Ph, R′ = H (87%)* 2e, R = Bn, R′ = H (81%) 2f, R = n-Bu, R′ = H (92%)* 2g, R = H, R′ = OMe (81%) 2h, R = Ph, R′ = OMe (84%) | 2i, R = H, R′ = H (84%) 2j, R = Ph, R′ = H (91%) 2k, R = Bn, R′ = H (82%) 2l, R = Bn, R′ = OMe (83%) 2m, R = Ph, R′ = OMe (87%) | 2n, R = H (92%) 2o, R = Ph (95%) 2p, R = n-Bu (93%) | | |
| 2q (74%) | 2r (87%) | 2s (89%) | 2t, R = H, (91%) 2u, R = OMe (94%) | |
| 2a′ (95%) | 2b′, X = NMe, R = Me (80%) 2c′, X = S, R = Me (81%) 2d′, X = NBn, n-Bu (84%) | 2e′, X = O (81%) 2f′, X = S (92%) | 2g′ (81%) | |
| 1v (X = H) 1w (X = Br) | 1x | 1y | 1z | 1h′ |

Accordingly, in another preferred embodiment, the invention encompasses α-substituted acryl aldehydes of Formula 2

wherein n represents 0, 1
“A” is selected from the group consisting of phenyl; benzyl; substituted or unsubstituted furyl, thiophenyl, pyrrolyl, pyridyl, benzofuryl, benzothiophenyl, indolyl, wherein substituents are selected from N—(C₁-C₄)alkyl or N-arylalkyl;
R is selected from the group consisting of hydrogen, aryl, arylalkyl, (C₁-C₆) linear or branched alkyl;
R¹, R² and R³ are similar or different and independently selected from the group consisting of hydrogen, (C₁-C₄)alkoxy, N—(R⁴)₂, where R⁴ is (C₁-C₆)alkyl;
with a proviso when n is 0, then A is selected from the group consisting of benzocyclohexyl and ferrocenyl.

The representative α-substituted acryl aldehydes of Formula 2 according to the invention comprise the following compounds.
(i) 2-(4-Methoxybenzyl)acrylaldehyde;
(ii) 2-((4-Methoxyphenyl)(phenyl)methyl)acrylaldehyde;
(iii) 3-(4-Methoxyphenyl)-2-methylene-4-phenylbutanal;
(iv) 3-(4-Methoxyphenyl)-2-methyleneheptanal;
(v) 2-(2,4-Dimethoxybenzyl)acrylaldehyde;
(vi) 2-((2,4-Dimethoxyphenyl)(phenyl)methyl)acrylaldehyde;
(vii) 2-(3,4-Dimethoxybenzyl)acrylaldehyde;
(viii) 2-((3,4-Dimethoxyphenyl)(phenyl)methyl)acrylaldehyde;
(ix) 3-(3,4-Dimethoxyphenyl)-2-methylene-4-phenylbutanal;
(x) 2-Methylene-4-phenyl-3-(3,4,5-trimethoxyphenyl)butanal;
(xi) 2-(Phenyl(3,4,5-trimethoxyphenyl)methyl)acrylaldehyde;
(xii) 2-(4-(Dimethylamino)benzyl)acrylaldehyde;
(xiii) 2-((4-(Dimethylamino)phenyl)(phenyl)methyl)acrylaldehyde;
(xiv) 3-(4-(Dimethylamino)phenyl)-2-methyleneheptanal;
(xv) 2-((3,5-Dimethoxyphenyl)(phenyl)methyl)acrylaldehyde;
(xvi) 2-Benzhydrylacrylaldehyde;
(xvii) 2-(6-Methoxy-1,2,3,4-tetrahydronaphthalen-1-yl)acrylaldehyde′
(xviii) 2-((4-Methoxyphenyl)(phenyl)methyl)-3-phenylacrylaldehyde;
(xix) 2-((2,4-Bimethoxyphenyl)(phenyl)methyl)-3-phenylacrylaldehyde;
(xx) Cyclopenta-2,4-dien-1-yl(2-(2-formylallyl) cyclopenta-2,4-dien-1-yl)iron;
(xxi) 3-(1-Methyl-1H-pyrrol-2-4)-2-methylenebutanal;
(xxii) 2-Methylene-3-(thiophen-2-yl)butanal;
(xxiii) 3-(1-Benzyl-1H-pyrrol-2-4)-2-methyleneheptanal;
(xxiv) 3-(Benzofuran-2-yl)-2-methylenebutanal;
(xxv) 3-(Benzo [b]thiophen-2-yl)-2-methylenebutanal;
(xxvi) 3-(1-Benzyl-1H-indol-3-yl)-2-methyleneheptanal;

All the reactions as per the table 3 were carried out by employing 0.05 mol % of the catalyst along with 0.15 mol % of silver salt as an additive to achieve the compounds of Formula 2 with good yields as detailed herein below.

The C₁-secondary allenyl ethers of the (4-methoxyphenyl)methanol with n-butyl 1d (on 1 g scale), phenyl 1e (1 g scale) and benzyl 1f substitutions underwent the [1,3] rearrangement smoothly and provided the corresponding acryl aldehydes 2d-2f in excellent yields.

A similar trend was observed with the substrates having methoxy group(s) at either ortho and/or meta and/or para (2g-2m and 2q) positions, which revealed that the presence of the methoxy substituent is important, but that it is not necessary for the substituent to be at particular position. Similarly, the [1,3] rearrangement of allenyl ether of electron-rich 6-methoxy-1,2,3,4-tetrahydronaphthalen-1-ol (1s) was facile.

The rearrangement of the (4-(N,N-dimethylamino)phenyl)-methanol allenyl ethers 1n-1p also proceeded smoothly and delivered the corresponding rearranged products 2n-2p in 92-95% yield. Although the simple benzyl allenyl ethers 1b and 1v are not compatible, gratifyingly, the diphenylmethanolallenyl ether 1r gave the rearranged product 2r in 87% yield, revealing that the stabilization of the intermediate carbocation is important. However, as expected upon the stabilization of the intermediate carbocation, the rearrangement of allenyl ethers of 1-(naphthalen-2-yl)ethan-1-ol 1x, 1-(naphthalen-1-yl)ethan-1-ol 1y and (tetrahydrofuran-2-yl)methanol 1z was found to be unsuccessful.

The successful synthesis of the 2,3-disubstituted acryl aldehydes 2t and 2u (isolated as an inseparable E/Z mixture) reveals the applicability of this methodology for the [1,3] rearrangement of the C1-substituted allenyl ethers. In yet another embodiment, the feasibility of the rearrangement with other electron rich and heterocyclic systems was examined. As shown in Table 3, the rearrangement of ferrocenyl methanol allenyl ether 1a′ provided the corresponding acrylaldehyde 2a.′. The [1,3] rearrangement of allenylethers having various heterocyclic units was also found to be facile under these conditions. The acryl aldehydes having N-methylpyrrole (2b), N-benzylpyrrole (2d), thiophene (2c′), benzofuran (2e′), benzothiophene (2f′) and N-benzylindole (2g′) structural units have been prepared in excellent yields by employing 0.05 mol % of the catalyst. However, under these conditions, the pyridine derived allenyl ether 1h′ was found to remain intact.

In yet another embodiment, the mechanism of the reaction is assessed based on two possible modes for the activation of allenyl ether-through either (i) coordination with the oxygen; or (ii) formation of η¹ complex A via the p-complexation with the electron rich olefin of the allene unit.

In the case of the reaction of 1e with Au(PPh₃)NO₃, it has been proposed by Cui and co-workers that the mechanism operates through the formation of an η¹ complex (scheme III).

In yet another embodiment, the invention demonstrates that the electrophilicity of the Au[I] complex is important to promote the cleavage of the carbinol C—O bond leading to the [1,3] rearrangement by conducting controlled experiments. According to this embodiment, when allenyl ether 1c was exposed to Au(PPh3)SbF₆ in the presence of 3 equivalents of methanol, the methyl PMB ether 3 was obtained exclusively without any traces of the rearranged product 2c or of the allylicacetal resulting from hydroalkoxylation with methanol (Scheme IV).

The complementary result thus obtained reveals that the electrophilicity of the Au[I] complex is important and suggests the possibility of the reaction proceeding through coordination of gold(I) to the lone pair of oxygen as depicted in scheme III. This coordination leads to significant elongation of the carbinol C—O bond and it depends strongly on the electrophilicity of the substituent attached to the oxygen. More electrophilic substituents promote the cleavage of the carbinol C—O bond leading to the [1,3] rearrangement. On the other hand, the less electrophilic substituents disfavour the cleavage of the C—O bond, which was the case with the substrates 1a, 1b, 1v-1z, where the hydrolysis of the C—O bond occurred through the allenyl ether activation by the [Au]-complex.

Thus the present invention provides hither to unexploited [Au]-catalysed [1,3] O-C rearrangement of allenyl ethers leading to the C₂-substituted acryl aldehydes The reaction is facile even with 5×10⁻² mol % of catalyst, which is the lowest catalyst loading that has been reported in the area of homogeneous gold-catalysis.

The following examples are given for illustration of the method of preparation of α-substituted acryl aldehydes via a gold-catalysed [1,3] rearrangement of the allenyl ethers. It should be noted that the present disclosure is not limited to the specific details embodied in the examples.

### EXAMPLES

Reactions were carried out in anhydrous solvents under an atmosphere of argon in oven-dried glassware. NMR spectra were recorded on JEOL AL-400 (400 MHz), Bruker AC 200 MHz, Bruker DRX 400 MHz and Bruker DRX 500 MHz spectrometers, and TMS was used as an internal standard of spectrometers. The chemical shifts were reported in parts per million (6) relative to internal standard TMS (0 ppm) and for CDCl₃ (7.25 ppm). The peak patterns are indicated as follows: s, singlet; d, doublet; dd, doublet of doublet; t, triplet; m, multiplet; q, quartet. The coupling constants, J, are reported in Hertz (Hz). Mass spectroscopy was carried out on PI QStar Pulsar (Hybrid Quadrupole-TOF LC/MS/MS) and High-resolution mass spectra (HRMS) were recorded on a Thermo Scientific Q-Exactive, Accela 1250 pump, and IR spectra were recorded on FT-IR PerkinElmer spectrometer by neat for oil sample and a CH₃C1 solution for solid samples. Column chromatography was performed over silica gel 100-200 mesh. All reagents were weighed and handled in air and backfilled under argon at room temperature. Unless otherwise noted, all reactions were performed under an argon atmosphere. All reagents were purchased from Aldrich and Alfa Easer and used without further purification. Compounds 1a-1z, and 1a′-1h′ are prepared following the procedures reported.

### Example 1

General Procedure for Synthesis of allenol ethers as per the procedure disclosed in B. M. Trost, J. Xie, J. Am. Chem. Soc. 2006, 128, 6044:

At room temperature, a solution of propargyl ether (2 mmol) in THF (10 mL) was treated with KOtBu (0.5 mmol) and the resulting suspension was stirred at room temperature for 1 h before quenching it with ice water. The contents were portioned between ethyl acetate (20 mL) and water (20 mL). The organic layer was separated and the aqueous layer was extracted with (2×10 ml) ethyl acetate. The combined organic layer was concentrated under reduced pressure and the crude was subjected for the next step. For analytical purpose, the crude was purified by flash chromatography.

Using the above procedure the following representative compounds of Formula 1a to 1z and 1a′ to 1h′ as depicted in table 4, were prepared.

TABLE 4

1, 70-75%

| 1a, 79% | 1b, 82% | 1c, 83% | 1d, 85% | 1e, 76% |
|---|---|---|---|---|
| 1f, 85% | 1g, 77% | 1h, 79% | 1i, 78% | 1j, 81% |
| 1k, 78% | 1l,75% | 1m, 82% | 1n, 83% | 1o, 79% |
| 1p, 86% | 1a, 84% | 1r, 81% | 1s, 76% | 1t*, 81% |
| 1u*, 80% | 1v, 81% | 1w, 83% | 1x, 72% | 1y, 70% |
| 1z*, 73% | 1a′, 89% | 1b′, 76% | 1c′, 84% | 1d′, 73% |
| 1e′, 83% | 1f′, 84% | 1g′, 81% | 1h′, 72% | |

### ((propa-1,2-dien-1-yloxy)methyl)benzene (1b)

Light yellowish oily liquid; 82%; (R_{f}=0.7, 5% ethyl acetate/pet. ether); ¹H NMR (200 MHz, CDCl₃): δ 4.60 (s, 2H), 5.46 (d, J=5.9 Hz, 2H), 6.83 (t, J=5.9 Hz, 1H), 7.22-7.43 (m, 5H) ppm.

### 1-Methoxy-4-((propa-1,2-dien-1-yloxy)methyl)benzene(1c)

Colorless syrup; 83%; (R_{f}=0.5, 5% ethyl acetate/pet. ether); ¹H NMR (200 MHz, CDCl₃): δ 3.79 (s, 1H), 4.53 (s, 1H), 5.46 (d, J=5.9 Hz, 2H), 6.80 (t, J=5.9 Hz, 1H), 6.85-6.91 (m, 2H), 7.27 (d, J=8.6 Hz, 1H); ¹³C NMR (50 MHz, CDCl₃): δ 55.2 (q), 70.3 (d), 90.9 (d), 113.8 (d, 2C), 121.4 (d), 129.3 (s), 129.4 (d), 129.5 (d), 159.3 (s), 201.3 (s) ppm.

### 1-Methoxy-4-(phenyl(propa-1,2-dien-1-yloxy)methyl)benzene(1d)

Yellow color oil; 85%; (R_{f}=0.7, 5% ethyl acetate/pet. ether); ¹H NMR (200 MHz, CDCl₃): δ 3.82 (s, 3H), 5.34 (s, 1H), 5.35-5.42 (m, 1H), 5.78 (s, 1H), 6.83 (t, J=5.9 Hz, 1H), 6.87-6.96 (m, 2H), 7.26-7.35 (m, 3H), 7.35-7.43 (m, 4H); ¹³C NMR (50 MHz, CDCl₃): δ 55.1 (q), 81.5 (d), 90.6 (t), 113.7 (d, 2C), 120.4 (d), 126.8 (d, 2C), 127.4 (d), 128.3 (d, 2C), 128.4 (d, 2C), 133.5 (s), 141.5 (s), 159.0 (s), 202.0 (s) ppm.

### 1-Methoxy-4-(1-(propa-1,2-dien-1-yloxy)pentyl)benzene (1f)

Yellow color syrup; 85%; (R_{f}=0.7, 5% ethyl acetate/pet. ether); ¹H NMR (200 MHz, CDCl₃): δ 0.82-0.95 (m, 3H), 1.19-1.46 (m, 5H), 1.59-1.83 (m, 1H), 1.83-2.07 (m, 1H), 3.82 (s, 3H), 4.57 (t, J=6.7 Hz, 1H), 5.15-5.40 (m, 2H), 6.61 (t, J=5.9 Hz, 1H), 6.84-6.96 (m, 2H), 7.18-7.28 (m, 2H); ¹³C NMR (50 MHz, CDCl₃): δ 13.9 (q), 22.5 (t), 27.8 (t), 37.2 (t), 55.1 (q), 80.7 (d), 89.8 (t), 113.6 (d, 2C), 120.2 (d), 127.8 (d, 2C), 134.2 (s), 158.9 (s), 202.2 (s) ppm.

### 2,4-dimethoxy-1-(phenyl(propa-1,2-dien-1-yloxy)methyl)benzene (1h)

Yellow color gum; 79%; (R_{f}=0.5, 5% ethyl acetate/pet. ether); ¹H NMR (200 MHz, CDCl₃): δ 3.83 (d, J=1.3 Hz, 6H), 5.30 (d, J=1.5 Hz, 1H), 5.33 (d, J=1.5 Hz, 1H), 6.16 (s, 1H), 6.46-6.60 (m, 2H), 6.75-6.86 (m, 1H), 7.28-7.48 (m, 6H);

### 1,2-Dimethoxy-4-(phenyl(propa-1,2-dien-1-yloxy)methyl)benzene (1j)

Yellow color gum; 78%; (R_{f}=0.6, 5% ethyl acetate/pet. ether); ¹H NMR (200 MHz, CDCl₃): δ 3.83 (d, J=2.0 Hz, 6H), 5.29 (dd, J=6.0, 0.9 Hz, 2H), 5.69 (s, 1H), 6.75 (t, J=6.0 Hz, 1H), 6.79-6.88 (m, 3H), 7.21-7.34 (m, 5H); ¹³C NMR (50 MHz, CDCl₃): δ 55.8 (q, 2C), 81.7 (d), 90.6 (t), 110.2 (d), 110.8 (d), 119.7 (d), 120.4 (d), 126.9 (d, 2C), 127.5 (d), 128.3 (d, 2C), 133.8 (s), 141.4 (s), 148.5 (s), 148.9 (s), 202.0 (s) ppm.

### 1,2,3-Trimethoxy-5-(phenyl(propa-1,2-dien-1-yloxy)methyl)benzene (1m)

Yellow color gum; 82%; (R_{f}=0.4, 5% ethyl acetate/pet. ether); ¹H NMR (200 MHz, CDCl₃): δ 3.80-3.86 (m, 9H), 5.26-5.41 (m, 2H), 5.69 (s, 1H), 6.59 (s, 2H), 6.80 (t, J=6.0 Hz, 1H), 7.24-7.40 (m, 5H); ¹³C NMR (100 MHz, CDCl₃): δ 55.9 (q, 2C), 60.7 (q), 81.8 (d), 90.7 (t), 103.9 (d, 2C), 120.3 (d), 126.8 (d, 2C), 127.6 (d), 128.2 (d, 2C), 136.8 (s), 137.1 (s), 141.0 (s), 153.0 (s, 2C), 201.8 (s) ppm.

### 1,3-dimethoxy-5-(phenyl(propa-1,2-dien-1-yloxy)methyl)benzene (1q)

Yellow color gel; 84%; (R_{f}=0.5, 5% ethyl acetate/pet. ether); ¹H NMR (200 MHz, CDCl₃): δ 3.80 (s, 6H), 5.37 (d, J=6.0 Hz, 2H), 5.74 (s, 1H), 6.42 (t, J=2.3 Hz, 1H), 6.59 (d, J=2.3 Hz, 2H), 6.85 (t, J=5.9 Hz, 1H), 7.28-7.45 (m, 5H); ¹³C NMR (50 MHz, CDCl₃): δ 55.2 (q, 2C), 81.8 (d), 90.8 (t), 99.3 d), 105.0 (d, 2C), 120.4 (d), 126.9 (d, 2C), 127.6 (d), 128.3 (d, 2C), 141.1 (s), 143.7 (s), 160.7 (s, 2C), 201.9 (s) ppm;

### 2-(Octa-1,2-dien-4-yl)pyridine(1h′)

Black color oil; 72%; (R_{f}=0.5, 5% ethyl acetate/pet. ether); ¹H NMR (200 MHz, CDCl₃): δ 0.79-0.92 (m, 3H), 1.21-1.47 (m, 4H), 1.76-1.94 (m, 2H), 4.73 (t, J=6.4 Hz, 1H), 5.10 (dd, J=8.3, 5.9 Hz, 1H), 5.3 (dd, J=8.3, 6.0 Hz, 1H), 6.70 (t, J=5.9 Hz, 1H), 7.1 (ddd, J=7.5, 4.9, 1.1 Hz, 1H), 7.3 (d, J=7.8 Hz, 1H), 7.60-7.70 (m, 1H), 8.5-8.6 (m, 1H); ¹³C NMR (100 MHz, CDCl₃): δ 13.9 (q), 22.5 (t), 27.5 (t), 36.0 (t), 81.5 (d), 90.5 (t), 120.4 (d), 120.5 (d), 122.0 (d), 136.4 (d), 148.9 (d), 161.7 (s), 201.7 (s) ppm.

### Example 2

### General Procedure for Synthesis of α-Substituted Acryl Aldehydes

At 0° C., to a solution of allenylether 1c (1.0 g, 5.68 mmol) in anhydrous CH₂Cl₂ (100 ml) was added above catalyst solution [(2.80 ml, 2.83 mmol) prepared by dissolving Au(PPh₃)Cl (5 mg, 10.1 mmol) and AgSbF₆ (10 mg, 29.1 μmol) in CH₂Cl₂ (10 ml)] and allowed to stir for 5 minutes. The reaction mixture was concentrated under reduced pressure and the crude was purified by column chromatography (100-200 Silicagel) to afford 2c (910 mg, 91%) as a colorless oil.

### Catalyst Optimization

Further, the molar ratio of catalyst and the additive kept at a ratio of 1:3 ([Au] Complex 5 mol % and additive 15 mol %) to obtain best turnover frequency.

Accordingly, the following α-substituted acryl aldehydes prepared in accordance with example 2 and characterized using ¹H NMR, ¹³C NMR, IR and

Mass spectroscopy as shown below.

### 2-(4-Methoxybenzyl)acrylaldehyde (2c)

Yellow oil; 91%; (R_{f}=0.6, 5% ethyl acetate/pet. ether); IR (CHCl₃) v: 3361, 2998, 2954, 2836, 2700, 1690, 1611, 1509, 1464, 1300, 1248, 1178, 1035, 958, 852, 809, 769 cm⁻¹; ¹H NMR (200 MHz, CDCl₃): δ 3.52 (s, 2H), 3.80 (s, 3H), 6.04-6.13 (m, 2H), 6.85 (m, 2H), 7.11 (m, 2H), 9.61 (s, 1H); ¹³C NMR (50 MHz, CDCl3): δ 33.3 (t), 55.2 (q), 113.9 (d, 2C), 130.1 (d, 2C), 135.0 (t), 150.1 (s, 2C), 158.2 (s), 194.1 (d) ppm; FIRMS (ESI+): calcd. For C₁₁H₁₂O₂Na [M+Na]⁺ 199.0730. found 199.0728.

### 2-((4-Methoxyphenyl)(phenyl)methyl)acrylaldehyde (2d)

Colorless syrup; 97%; (R_{f}=0.5, 5% ethyl acetate/pet. ether); IR (CHCl₃) v: 3367, 3027, 2836, 1692, 1609, 1509, 1463, 1302, 1250, 1177, 1033, 966, 844, 751, 701, 545 cm⁻¹; ¹H NMR (200 MHz, CDCl₃): δ 3.72 (s, 3H), 5.33 (s, 1H), 5.96 (d, J=1.1 Hz, 1H), 6.23 (s, 1H) 6.78-6.86 (m, 2H), 7.02 (d, J=8.8 Hz, 2H), 7.06-7.15 (m, 2H), 7.16-7.28 (m, 3H), 9.60 (s, 1H); ¹³C NMR (50 MHz, CDCl₃): δ 48.4 (d), 55.2 (q), 113.9 (d, 2C), 126.6 (d), 128.4 (d, 2C), 128.8 (d, 2C), 129.9 (d, 2C), 133.1 (s), 136.6 (t), 141.5 (s), 153.0 (s), 158.3 (s), 193.2 (d) ppm; HRMS (ESI+): calcd. For C₁₇H₁₆O₂Na [M+Na]⁺ 275.1043. found 275.1039.

### 3-(4-Methoxyphenyl)-2-methylene-4-phenylbutanal(2e)

Colorless syrup; 81%; (R_{f}=0.6, 10% ethyl acetate/pet. ether); IR (CHCl₃) v:

3367, 3027, 2836, 1692, 1609, 1509, 1463, 1302, 1250, 1177, 1033, 966, 844, 751, 701, 545 cm⁻¹; ¹H NMR (200 MHz, CDCl₃): δ 3.00-3.13 (m, 1H), 3.14-3.26 (m, 1H), 3.77 (s, 3H), 4.19 (t, J=7.9 Hz, 1H), 6.10 (s, 1H), 6.38 (d, J=0.9 Hz, 1H), 6.74-6.85 (m, 2H), 7.03-7.10 (m, 3H), 7.11-7.24 (m, 4H), 9.49 (s, 1H); ¹³C NMR (50 MHz, CDCl₃): δ 40.3 (t), 44.0 (d), 55.2 (q), 113.7 (d, 2C), 126.0 (d), 128.1 (d, 2C), 128.9 (d, 2C), 129.1 (d, 2C), 133.4 (s), 134.1 (t), 139.6 (s), 152.6 (s), 158.2 (s), 193.9 (d) ppm; HRMS (ESI+): calcd. For C₁₈H₁₈O₂Na [M+Na]⁺ 289.1199. found 289.1195.

### 3-(4-Methoxyphenyl)-2-methyleneheptanal (2f)

Yellow gum; 92%; (Rf=0.5, 10% ethyl acetate/pet. ether); IR (CHCl₃) v: 3366, 2956, 2931, 1693, 1610, 1509, 1464, 1301, 1248, 1178, 1036, 942, 827 cm⁻¹; ¹H NMR (200 MHz, CDCl₃): δ 0.86 (t, J=6.9 Hz, 3H), 1.12-1.41 (m, 4H), 1.70-1.88 (m, 2H), 3.78 (s, 3H), 3.80-3.86 (m, 1H), 6.05 (s, 1H), 6.30 (s, 1H), 6.79-6.88 (m, 2H), 7.09-7.19 (m, 2H), 9.51 (s, 1H); ¹³C NMR (50 MHz, CDCl₃): δ 13.9 (q), 22.5 (t), 29.8 (t), 33.8 (t), 42.0 (d), 55.1 (q), 113.7 (d, 2C), 128.9 (d, 2C), 133.3 (t), 134.4 (s), 153.7 (s), 158.1 (s), 194.0 (d) ppm; HRMS (ESI+): calcd. For C₁₅H₂₀O₂Na [M+Na]⁺255.1356. found 255.1352.

### 2-(2,4-Dimethoxybenzyl)acrylaldehyde (2g)

Yellow gum; 91%; (R_{f}=0.6, 5% ethyl acetate/pet. ether); IR (CHCl₃) v: 3359, 2998, 2935, 2835, 1909, 1690, 1590, 1512, 1464, 1418, 1333, 1262, 1155, 1029, 954, 867, 805, 771, 755 cm⁻¹; ¹H NMR (200 MHz, CDCl₃): δ 3.49 (s, 2H), 3.78 (s, 3H), 3.81 (s, 3H), 5.96-6.04 (m, 2H), 6.41-6.48 (m, 2H), 7.02 (s, 1H), 9.61 (s, 1H); ¹³C NMR (100 MHz, CDCl₃): δ 27.8 (t), 55.3 (q, 2C), 98.6 (d), 103.9 (d), 118.8 (s), 131.1 (d), 134.7 (t), 149.2 (s), 158.3 (s), 159.7 (s), 194.4 (d) ppm; HRMS (ESI+): calcd. For C₁₂H₁₄O₃Na [M+Na]⁺ 229.0835. found 229.0833.

### 2-((2,4-Dimethoxyphenyl)(phenyl)methyl)acrylaldehyde (2h)

Yellow gum; 94%; (R_{f}=0.6, 5% ethyl acetate/pet. ether); IR (CHCl₃) v: 3367, 3023, 2957, 2836, 1693, 1591, 1514, 1417, 1265, 1141, 1028, 954, 754, 701 cm⁻¹; ¹H NMR (200 MHz, CDCl₃): δ 3.80 (s, 3H), 3.84 (s, 3H), 5.69 (s, 1H), 5.98 (s, 1H), 6.28 (s, 1H), 6.41-6.49 (m, 1H), 6.54 (d, J=2.4 Hz, 1H), 6.81 (d, J=8.3 Hz, 1H), 7.15-7.23 (m, 2H), 7.33 (td, J=5.3, 1.8 Hz, 3H), 9.70 (s, 1H); ¹³C NMR (100 MHz, CDCl₃): δ 42.1 (d), 55.2 (q), 55.4 (q), 98.7 (d), 103.6 (d), 122.4 (s), 126.3 (d), 128.2 (d, 2C), 128.9 (d, 2C), 129.6 (d), 135.7 (t), 141.0 (s), 152.6 (s), 157.6 (s), 159.6 (s), 193.2 (d) ppm; HRMS (ESI+): calcd. For C₁₈H₁₈O₃Na [M+Na]⁺ 305.1148. found 305.1145.

### 2-(3,4-Dimethoxybenzyl)acrylaldehyde (2i)

Yellow oil; 94%; (R_{f}=0.4, 5% ethyl acetate/pet. ether); IR (CHCl₃) v: 3359, 2998, 2935, 2835, 1909, 1690, 1590, 1512, 1464, 1418, 1333, 1262, 1155, 1029, 954, 867, 805, 771, 755 cm⁻¹; ¹H NMR (200 MHz, CDCl₃): δ 3.50 (s, 2H), 3.85 (s, 6H), 6.05 (d, J=0.8 Hz, 1H), 6.11 (s, 1H), 6.68-6.75 (m, 2H), 6.77-6.83 (m, 1H), 9.60 (s, 1H); ¹³C NMR (50 MHz, CDCl₃): δ 33.7 (t), 55.7 (q), 55.8 (q), 111.2 (d), 112.3 (d), 121.0 (d), 130.6 (s), 134.9 (t), 147.6 (s), 148.9 (s), 149.9 (s), 194.0 (d) ppm; HRMS (ESI+): calcd. For C₁₂H₁₄O₃Na [M+Na]⁺ 229.0835. found 229.0833.

### 2-((3,4-Dimethoxyphenyl)(phenyl)methyl)acrylaldehyde (2j)

Yellow gum; 91%; (R_{f}=0.6, 5% ethyl acetate/pet. ether); IR (CHCl₃) v: 3367, 3023, 2957, 2836, 1693, 1591, 1514, 1417, 1246, 1141, 1028, 954, 754, 701 cm⁻¹; 1H NMR (200 MHz, CDCl₃): δ 3.85 (s, 3H), 3.90 (s, 3H), 5.38 (s, 1H), 6.06 (d, J=1.0 Hz, 1H), 6.35 (s, 1H), 6.64-6.73 (m, 2H), 6.80-6.88 (m, 1H), 7.14-7.20 (m, 2H), 7.28-7.41 (m, 3H), 9.70 (s, 1H); ¹³C NMR (50 MHz, CDCl₃): δ 48.6 (d), 55.7 (q, 2C), 110.9 (d), 112.4 (d), 120.7 (d), 126.5 (d), 128.3 (d, 2C), 128.7 (d, 2C), 133.5 (s), 136.5 (t), 141.2 (s), 147.7 (s), 148.9 (s), 152.8 (s), 193.0 (d) ppm; HRMS (ESI+): calcd. For C₁₈H₁₈O₃Na [M+Na]⁺305.1148. found 305.1144.

### 3-(3,4-Dimethoxyphenyl)-2-methylene-4-phenylbutanal (2k)

Colorless gum; 82%; (R_{f}=0.6, 10% ethyl acetate/pet. ether); IR (CHCl₃) v: 3362, 3025, 2934, 2835, 1692, 1591, 1515, 1454, 1419, 1260, 1141, 1028, 950, 809, 757, 700 cm⁻¹; ¹H NMR (200 MHz, CDCl₃): δ 2.99-3.13 (m, 1H), 3.13-3.26 (m, 1H), 3.79 (s, 3H), 3.84 (s, 3H), 4.17 (t, J=7.9 Hz, 1H), 6.13 (s, 1H), 6.37-6.41 (m, 1H), 6.63 (s, 1H), 6.71-6.77 (m, 2H), 7.02-7.09 (m, 2H), 7.13-7.23 (m, 3H), 9.51 (s, 1H); ¹³C NMR (50 MHz, CDCl₃): δ 40.3 (t), 44.4 (d), 55.8 (q, 2C), 111.0 (d), 111.8 (d), 119.7 (d), 126.0 (d), 128.1 (d, 2C), 128.9 (d, 2C), 133.9 (s), 134.1 (t), 139.5 (s), 147.6 (s), 148.6 (s), 152.5 (s), 193.8 (d) ppm; HRMS (ESI+): calcd. For C₁₉H₂₀O₃Na [M+Na]⁺319.1305. found 319.1304.

### 2-Methylene-4-phenyl-3-(3,4,5-trimethoxyphenyl)butanal (21)

White gum; 83%; (R_{f}=0.6, 10% ethyl acetate/pet. ether); IR (CHCl₃) v: 3362, 2997, 2936, 2837, 1693, 1589, 1506, 1455, 1421, 1327, 1239, 1126, 1010, 700 cm⁻¹; ¹H NMR (200 MHz, CDCl₃): δ 2.98-3.13 (m, 1H), 3.13-3.26 (m, 1H), 3.78 (s, 6H), 3.81 (s, 3H), 4.16 (t, J=7.8 Hz, 1H), 6.15 (s, 1H), 6.35 (s, 2H), 6.41 (s, 1H), 7.03-7.10 (m, 2H), 7.14-7.23 (m, 3H), 9.52 (s, 1H); ¹³C NMR (101 MHz, CDCl₃): δ 40.3 (t), 45.0 (d), 56.1 (q, 2C), 60.8 (q), 105.1 (d, 2C), 126.2 (d), 128.2 (d, 2C), 128.9 (d, 2C), 134.5 (t), 136.6 (s), 137.0 (s), 139.4 (s), 152.1 (s), 153.0 (s, 2C), 193.9 (d) ppm; HRMS (ESI+): calcd. For C₂₀H₂₂O₄Na [M+Na]⁺349.1410. found 349.1404.

### 2-(Phenyl(3,4,5-trimethoxyphenyl)methyl)acryl aldehyde (2m)

Yellow gum; 87%; (R_{f}=0.5, 10% ethyl acetate/pet. ether); IR (CHCl₃) v: 3366, 2998, 2938, 1959, 1693, 1589, 1505, 1419, 1237, 1126, 1008, 960, 823, 702 cm⁻¹; ¹H NMR (200 MHz, CDCl₃): δ 3.81 (s, 6H), 3.87 (s, 3H), 5.36 (s, 1H), 6.07 (d, J=1.1 Hz, 1H), 6.35-6.38 (m, 3H), 7.13-7.20 (m, 2H), 7.28-7.40 (m, 3H), 9.70 (s, 1H); ¹³C NMR (50 MHz, CDCl₃): δ 49.2 (d), 56.0 (q, 2C), 60.8 (q), 106.1 (d, 2C), 126.7 (d), 128.4 (d, 2C), 128.8 (d, 2C), 136.6 (s), 136.7 (s), 136.8 (t), 140.9 (s), 152.6 (s), 153.1 (s, 2C), 193.1 (d) ppm; HRMS (ESI+): calcd. For C₁₉H₂₀O₄Na [M+Na]⁺335.1254. found 335.1248.

### 2-(4-(Dimethylamino)benzyl)acrylaldehyde (2n)

Yellow oil; 92%; (R_{f}=0.6, 10% ethyl acetate/pet. ether); IR (CHCl₃) v: 3358, 2916, 2802, 1885, 1686, 1615, 1523, 1479, 1347, 1163, 1061, 947, 857, 800 cm⁻¹; ¹H NMR (400 MHz, CDCl₃): δ 2.94 (s, 6H), 3.49 (s, 2H), 6.04 (d, J=0.9 Hz, 1H), 6.12 (s, 1H), 6.72 (m, J=8.7 Hz, 2H), 7.08 (m, J=8.7 Hz, 2H), 9.62 (s, 1H); ¹³C NMR (50 MHz, CDCl₃): δ 33.0 (t), 40.7 (q, 2C), 112.9 (d, 2C), 125.8 (s), 129.7 (d, 2C), 134.7 (t), 149.3 (s), 150.4 (s), 194.2 (d) ppm; HRMS (ESI+): calcd. For C₁₂H₁₆ON [M+H]⁺ 190.1226. found 190.1225.

### 2-((4-(Dimethylamino)phenyl)(phenyl)methyl)acrylaldehyde (2o)

Yellow gum; 95%; (R_{f}=0.5, 10% ethyl acetate/pet. ether); IR (CHCl₃) v: 3365, 3026, 2885, 2803, 2696, 1884, 1692, 1612, 1520, 1449, 1350, 1221, 1162, 1061, 948, 806, 761, 701 cm⁻¹; ¹H NMR (200 MHz, CDCl₃): δ 3.00 (s, 6H), 5.37 (s, 1H), 6.08 (s, 1H), 6.34 (s, 1H), 6.71-6.80 (m, 2H), 7.01-7.10 (m, 2H), 7.16-7.24 (m, 2H), 7.29-7.42 (m, 3H), 9.72 (s, 1H); ¹³C NMR (50 MHz, CDCl₃): δ 40.6 (q, 2C), 48.2 (d), 112.7 (d, 2C), 126.4 (d), 128.3 (d, 2C), 128.8 (d, 2C), 129.6 (d, 2C), 136.4 (t), 141.9 (s), 149.2 (s), 153.2 (s, 2C), 193.4 (d) ppm; HRMS (ESI+): calcd. For C₁₈H₂₀ON [M+H]⁺ 266.1539. found 266.1539.

### 3-(4-(Dimethylamino)phenyl)-2-methyleneheptanal (2p)

Yellow Syrup; 93%; (R_{f}=0.6, 10% ethyl acetate/pet. ether); IR (CHCl₃) v: 3363, 2955, 2929, 1693, 1614, 1520, 1445, 1348, 1223, 1163, 1061, 812 cm⁻¹; ¹H NMR (200 MHz, CDCl₃): δ 0.84 (t, J=6.9 Hz, 3H), 1.14-1.37 (m, 4H), 1.70-1.84 (m, 2H), 2.91 (s, 6H), 3.75 (t, J=7.6 Hz, 1H), 6.00 (s, 1H), 6.26 (s, 1H), 6.61-6.74 (m, 2H), 7.02-7.11 (m, 2H), 9.50 (s, 1H); ¹³C NMR (50 MHz, CDCl₃): δ 13.9 (q), 22.6 (t), 29.9 (t), 33.8 (t), 40.7 (q, 2C), 41.8 (d), 112.6 (d, 2C), 128.6 (d, 2C), 130.3 (s), 133.1 (t), 149.2 (s), 154.1 (s), 194.3 (d) ppm; HRMS (ESI+): calcd. For C₁₆H₂₄ON [M+H]⁺ 246.1852. found 246.1851.

### 2-((3,5-Dimethoxyphenyl)(phenyl)methyl)acrylaldehyde (2q)

Yellow liquid; 74%; (R_{f}=0.6, 10% ethyl acetate/pet. ether); IR (CHCl₃) v: 3367, 3023, 2957, 2836, 1693, 1591, 1514, 1417, 1265, 1141, 1028, 954, 754, 701 cm⁻¹; ¹H NMR (200 MHz, CDCl₃): δ 3.79 (s, 6H), 5.36 (s, 1H), 6.09 (d, J=1.0 Hz, 1H), 6.31-6.37 (m, 3H), 6.38 (d, J=2.1 Hz, 1H), 7.14-7.21 (m, 2H), 7.29-7.36 (m, 3H), 9.69 (s, 1H); ¹³C NMR (50 MHz, CDCl₃): δ 49.3 (d), 55.2 (q, 2C), 98.4 (d), 107.4 (d, 2C), 126.7 (d), 128.5 (d, 2C), 128.9 (d, 2C), 136.8 (t), 140.8 (s), 143.5 (s), 152.4 (s), 160.8 (s, 2C), 193.1 (d) ppm; HRMS (ESI+): calcd. For C₁₈H₁₈O₃Na [M+Na]⁺305.1148. found 305.1146.

### 2-Benzhydrylacrylaldehyde (2r)

Yellow gum; 87%; (R_{f}=0.6, 10% ethyl acetate/pet. ether); IR (CHCl₃) v: 3362, 2995, 2932, 1894, 1690, 1617, 1509, 1456, 1253, 1229, 1162, 1047, 940, 830 cm⁻¹; ¹H NMR (200 MHz, CDCl3): δ 5.43 (s, 1H), 6.05 (d, J=1.1 Hz, 1H), 6.36 (s, 1H), 7.12-7.21 (m, 4H), 7.28-7.41 (m, 6H), 9.70 (s, 1H); ¹³C NMR (50 MHz, CDCl3): δ 49.2 (d), 126.7 (d, 2C), 128.5 (d, 4C), 128.9 (d, 4C), 136.8 (t), 141.1 (s, 2C), 152.7 (s), 193.1 (d) ppm; HRMS (ESI+): calcd. For C₁₆H₁₄ONa [M+Na]⁺245.0937. found 254.0933.

### 2-(6-Methoxy-1,2,3,4-tetrahydronaphthalen-1-yl)acrylaldehyde (2s)

Yellow Syrup; 89%; (R_{f}=0.6, 10% ethyl acetate/pet. ether); IR (CHCl₃) v: 3362, 2995, 2932, 1894, 1690, 1608, 1500, 1456, 1253, 1226, 1162, 1038, 940, 830 cm⁻¹; ¹H NMR (200 MHz, CDCl₃): δ1.66-1.81 (m, 3H), 1.87-2.01 (m, 1H), 2.72-2.82 (m, 2H), 3.79 (s, 3H), 4.11 (br. s., 1H), 5.81 (s, 1H), 6.12 (s, 1H), 6.64-6.72 (m, 2H), 6.77-6.84 (m, 1H), 9.61 (s, 1H); ¹³C NMR (50 MHz, CDCl₃): δ 19.2 (t), 28.3 (t), 29.7 (t), 36.4 (d), 55.2 (q), 112.4 (d), 113.3 (d), 129.2 (s), 130.6 (d), 137.0 (t), 139.1 (s), 155.4 (s), 157.8 (s), 194.0 (d) ppm; HRMS (ESI+): calcd. For C₁₄H₁₆O₂Na [M+Na]⁺ 239.1043. found 239.1040.

### 2-((4-Methoxyphenyl)(phenyl)methyl)-3-phenylacrylaldehyde(2t)

Colorless syrup; 91%; (R_{f}=0.4, 10% ethyl acetate/pet. ether); ¹H NMR (400 MHz, CDCl₃): δ 3.8 (s, 3H), 5.62 (s, 1H), 6.90 (d, J=8.6 Hz, 2H), 7.14 (d, J=8.6 Hz, 2H), 7.18-7.26 (m, H), 7.28-7.48 (m, 9H), 9.98 (s, 1H); ¹³C NMR (100 MHz, CDCl₃): δ 49.4 (d), 55.2 (q), 79.4 (d), 113.7 (d), 113.9 (d), 126.5 (d), 127.0 (d), 127.1 (d), 127.2 (d), 127.2 (d), 128.3 (d), 128.4 (d), 128.5 (d), 128.6 (d), 129.1 (d), 129.2 (d), 129.9 (d), 130.1 (d), 133.7 (s), 133.9 (s), 142.1 (s), 142.7 (s), 144.6 (s), 148.3 (d), 158.2 (s), 158.9 (s), 192.0 (d) ppm; HRMS (ESI+): calcd. For C₂₃H₂₀O₂Na [M+Na]⁺ 351.1356. found 351.1354.

### 2-((2,4-Bimethoxyphenyl)(phenyl)methyl)-3-phenylacrylaldehyde (2u)

Colorless syrup; 94%; (R_{f}=0.4, 10% ethyl acetate/pet. ether); ¹H NMR (400 MHz, CDCl₃): δ 3.72 (s, 3H), 3.76 (s, 4H), 5.60 (s, 1H), 5.90 (s, 1H), 6.20 (s, 1H), 6.33-6.43 (m, 1H), 6.43-6.50 (m, 1H), 6.72 (d, J=8.6 Hz, 1H), 7.11 (d, J=7.1 Hz, 2H), 7.17-7.41 (m, 5H), 9.62 (s, 1H); ¹³C NMR (100 MHz, CDCl₃): δ 42.1 (d), 43.3 (d), 55.3 (q), 55.5 (q), 98.7 (d), 98.8 (d), 103.6 (d), 122.5 (s), 126.4 (d), 128.3 (d), 129.0 (d), 129.2 (d), 129.7 (d), 129.9 (d), 130.0 (d), 134.2 (s), 135.7 (t), 141.0 (s), 141.6 (s), 144.3 (s), 147.1 (d), 152.6 (s), 157.7 (s), 159.7 (s), 191.9 (s), 193.3 (d) ppm; HRMS (ESI+): calcd. For C₂₄H₂₂O₃Na [M+Na]⁺ 381.1461. found 381.1461.

### Cyclopenta-2,4-dien-1-yl(2-(2-formylallyl)cyclopenta-2,4-dien-1-yl)iron (2a′)

Orange color powder; 96%; (R_{f}=0.6, 5% ethyl acetate/pet. ether); IR (CHCl₃) v: 3362, 3093, 2924, 2853, 1894, 1693, 1627, 1464, 1340, 1245, 1105, 959, 819 cm⁻¹; ¹H NMR (200 MHz, CDCl₃): δ 3.31 (bs, 2H), 4.06-4.20 (m, 9H), 5.95 (s, 1H), 6.08 (s, 1H), 9.56 (s, 1H); ¹³C NMR (50 MHz, CDCl₃): δ 28.1 (t), 67.7 (d, 2C), 68.4 (d), 68.7 (d, 3C), 68.9 (d, 2C), 69.4 (d), 84.6 (s), 134.6 (t), 150.2 (s), 194.0 (d) ppm; HRMS (ESI+): calcd. For C₁₄H₁₄OFe [M⁺] 254.0389. found 254.0386.

### 3-(1-Methyl-1H-pyrrol-2-yl)-2-methylenebutanal (2b′)

Yellow oil; 89%; (R_{f}=0.5, 5% ethyl acetate/pet. ether); ¹H NMR (200 MHz, CDCl₃): δ 1.40 (d, J=7.1 Hz, 3H), 3.39 (s, 3H), 4.04 (q, J=7.1 Hz, 1H), 5.95 (s, 1H), 6.02-6.04 (m, 1H), 6.04-6.07 (m, 1H), 6.07-6.12 (m, 1H), 6.54-6.60 (m, 1H), 9.63 (s, 1H); ¹³C NMR (100 MHz, CDCl₃): δ 19.6 (q), 28.9 (d), 33.4 (q), 106.1 (d), 106.5 (d), 121.8 (d), 134.4 (s), 134.8 (t), 154.0 (s), 193.7 (d) ppm; HRMS (ESI+): calcd. For C₁₀H₁₄ON [M+1]⁺ 164.1070. found 164.1070; C₁₀H₁₃ONNa [M+Na]⁺186.0889. found 186.0889.

### 2-Methylene-3-(thiophen-2-yl)butanal(2c′)

Colorless oil; 91%; (R_{f}=0.5, 5% ethyl acetate/pet. ether); ¹H NMR (200 MHz, CDCl₃): δ 1.52 (d, J=7.2 Hz, 3H), 4.34 (q, J=7.2 Hz, 1H), 6.09 (s, 1H), 6.28 (d, J=1.0 Hz, 1H), 6.86 (dt, J=3.4, 1.1 Hz, 1H), 6.94 (dd, J=5.12, 3.5 Hz, 1H), 7.16 (dd, J=5.1, 1.3 Hz, 1H), 9.58 (s, 1H); ¹³C NMR (100 MHz, CDCl₃): δ 21.4 (q), 32.5 (d), 123.5 (d), 124.1 (d), 126.6 (d), 134.2 (t), 147.4 (s), 154.2 (s), 193.4 (d) ppm; HRMS (ESI+): calcd. For C₉H₁₀ONaS [M+Na]⁺189.0345. found 189.0344.

### 3-(1-Benzyl-1H-pyrrol-2-yl)-2-methyleneheptanal (2d′)

Yellow oil; 84%; (R_{f}=0.5, 5% ethyl acetate/pet. ether); ¹H NMR (200 MHz, CDCl₃): δ 0.78-0.88 (m, 3H), 1.11-1.33 (m, 4H), 1.62-1.74 (m, 2H), 3.91 (t, J=7.3 Hz, 1H), 4.97 (s, 2H), 5.95 (s, 1H), 6.1 (s, 1H), 6.12-6.18 (m, 1H), 6.20-6.27 (m, 1H), 6.67-6.74 (m, 1H), 7.1 (dd, J=7.4, 1.7 Hz, 2H), 7.27-7.39 (m, 3H), 9.49 (s, 1H); ¹³C NMR (100 MHz, CDCl₃): δ 13.9 (q), 22.4 (t), 29.9 (t), 33.6 (d), 34.7 (t), 50.5 (t), 106.6 (d), 107.0 (d), 121.4 (d), 126.7 (d, 2C), 127.3 (d), 128.5 (d, 2C), 133.7 (s), 135.4 (t), 138.2 (s), 153.0 (s), 193.8 (d) ppm; HRMS (ESI+): calcd. For C₁₉H₂₄ON [M+1]⁺ 282.1852. found 282.1851; C₁₉H₂ONNa [M+Na]⁺ 304.1672. found 304.1671.

### 3-(Benzofuran-2-yl)-2-methylenebutanal (2e′)

Yellow gum; 91%; (R_{f}=0.4, 5% ethyl acetate/pet. ether); ¹H NMR (500 MHz, CDCl₃): δ 1.53 (d, J=7.0 Hz, 3H), 4.28 (d, J=7.0 Hz, 1H), 6.14 (s, 1H), 6.30 (s, 1H), 6.52 (s, 1H), 7.18-7.28 (m, 2H), 7.43 (d, J=7.9 Hz, 1H), 7.53 (d, J=7.3 Hz, 1H), 9.62 (s, 1H); ¹³C NMR (125 MHz, CDCl₃): δ 18.0 (q), 31.5 (d), 102.6 (d), 110.9 (d), 120.5 (d), 122.5 (d), 123.6 (d), 128.4 (s), 134.8 (t), 151.5 (s), 154.7 (s), 159.6 (s), 193.1 (d) ppm; HRMS (ESI+): calcd. For C₁₃H₁₃O₂ [M+1]⁺201.0910. found 201.0910.

### 3-(Benzo[b]thiophen-2-yl)-2-methylenebutanal (2f′)

Colorless gum; 92%; (R_{f}=0.4, 5% ethyl acetate/pet. ether); ¹H NMR (200 MHz, CDCl₃): δ 1.6 (d, J=7.1 Hz, 3H), 4.5 (d, J=7.1 Hz, 1H), 6.01-6.15 (m, 2H), 7.27 (d, J=1.0 Hz, 1H), 7.32-7.42 (m, 2H), 7.53-7.62 (m, 1H), 7.84-7.96 (m, 1H), 9.70 (s, 1H); ¹³C NMR (100 MHz, CDCl₃): δ 19.5 (q), 30.9 (d), 121.8 (d), 122.0 (d), 122.8 (d), 123.9 (d), 124.4 (d), 134.8 (t), 137.9 (s), 138.5 (s), 140.5 (s), 153.5 (s), 193.7 (d) ppm; HRMS (ESI+): calcd. For C₁₃H₁₂ONaS [M+Na]⁺239.0501. found 239.0500.

### 3-(1-Benzyl-1H-indol-3-yl)-2-methyleneheptanal(2g′)

Yellow gum; 81%; (R_{f}=0.4, 5% ethyl acetate/pet. ether); ¹H NMR (400 MHz, CDCl₃): δ 0.83-0.93 (m, 3H), 1.29-1.42 (m, 4H), 1.84-2.02 (m, 2H), 4.22 (t, J=7.6 Hz, 1H), 5.32 (s, 2H), 6.05 (s, 1H), 6.27 (s, 1H), 7.03 (s, 1H), 7.05-7.12 (m, 3H), 7.16 (ddd, J=8.2, 6.9, 1.4 Hz, 1H), 7.25 (d, J=8.2 Hz, 1H), 7.27-7.34 (m, 2H), 7.54 (dd, J=8.9, 1.1 Hz, 1H), 9.63 (s, 1H); ¹³C NMR (100 MHz, CDCl₃): δ 14.0 (q), 22.6 (t), 30.2 (t), 33.8 (t), 34.3 (d), 49.9 (t), 109.7 (d), 116.6 (s), 119.0 (d), 119.5 (d), 121.8 (d), 126.1 (d), 126.5 (d, 2C), 127.4 (d), 127.5 (d), 128.7 (d, 2C), 134.4 (t), 136.8 (s), 137.7 (s), 153.5 (s, 2C), 194.3 (d) ppm; HRMS (ESI+): calcd. For C₂₃H₂₆ON [M+1]⁺ 332.2009. found 232.2008; C₂₃H₂₅ONNa [M+Na]⁺ 354.1828. found 354.1828.

### Example 3

### Trapping of the Intermediate with Alcohols to Obtain Corresponding Alkoxy Compounds

General Procedure: At 0° C., a solution of allenylether 1c (100 mg, 567 δ mol) and 3 equivalents of nucleophile (MeOH) in dichloromethane (5 mL) was treated with the catalyst Stock solution (1 mol % catalyst) and stirred for 3 h at room temperature. The reaction mixture was concentrated under reduced pressure and the resulting crude was purified by silica gel column chromatography to afford 3 (71 mg, 81% yield) as a colorless liquid.

By using the above procedure, the following compounds 4 and 5 are prepared and characterized

### 1-Methoxy-4-(methoxy(phenyl)methyl)benzene (4)

Yellow color liquid, 85%; ¹H NMR (200 MHz, CDCl₃): δ 3.41 (s, 3H), 3.78 (s, 3H), 5.26 (s, 1H), 6.84-6.97 (m, 2H), 7.23-7.46 (m, 7H); ¹³C NMR (100 MHz, CDCl₃): δ 55.0 (q), 56.7 (q), 84.8 (d), 113.6 (d, 2C), 126.7 (d, 2C), 127.2 (d), 128.1 (d, 2C), 128.2 (d, 2C), 134.2 (s), 142.3 (s), 158.8 (s) ppm;

### 1-Methoxy-4-(1-(pent-4-yn-1-yloxy)pentyl)benzene (5)

Yellow oil; 72%; (R_{f}=0.5, 10% ethyl acetate/pet. ether); IR (neat) v: 3296, 2999, 2954, 2931, 1658, 1510, 1462, 1244, 1171, 1097, 1035, 830, 634 cm⁻¹; ¹H NMR (200 MHz, CDCl₃): δ 0.88 (t, J=6.8, 3H), 1.24-1.37 (m, 5H), 1.76 (t, J=6.4 Hz, 3H), 1.91 (t, J=2.6 Hz, 1H), 2.23-2.35 (m, 2H), 3.25-3.44 (m, 2H), 3.82 (s, 3H), 4.13 (dd, J=6.3, 7.1 Hz, 1H), 6.88 (d, J=8.6 Hz, 2H), 7.20 (d, J=8.6 Hz, 2H); ¹³C NMR (100 MHz, CDCl₃): δ 14.0 (q), 15.4 (t), 22.6 (t), 28.1 (t), 28.8 (t), 38.0 (t), 55.2 (q), 66.8 (t), 68.3 (s), 82.0 (d), 84.1 (d), 113.6 (d, 2C), 127.7 (d, 2C), 135.1 (s), 158.8 (s) ppm; HRMS (ESI+): calcd. For C₁₇H₂₄O₂Na [M+Na]⁺283.1668. found 283.1669.

1. A process for the preparation of α-substituted acryl aldehydes of Formula 2
wherein n represents 0,1
“A” is selected from the group consisting of phenyl; benzyl; substituted or unsubstituted furyl, thiophenyl, pyrrolyl, pyridyl, benzofuryl, benzothiophenyl, indolyl, wherein substituents are selected from N—(C₁-C₄)alkyl or N-arylalkyl;
R is selected from the group consisting of hydrogen, aryl, arylalkyl, (C₁-C₆) linear or branched alkyl;
R¹, R² and R³ are similar or different and independently selected from the group consisting of hydrogen, (C₁-C₄)alkoxy, N—(R⁴)₂, where R⁴ is (C₁-C₆)alkyl;
with a proviso when n is 0, then “A” is selected from the group consisting of benzocyclohexyl and ferrocenyl;
comprising subjecting allenyl ethers of Formula 1 to Au(I) complex catalysed [1, 3] O→C rearrangement to obtain α-substituted acryl aldehydes of Formula 2 with turnover frequency of 4600 h⁻¹.

2. The process according to claim 1, wherein the Au(I) complex is selected from the group consisting of AuCl₃; AuBr₃, AuCl(PPh₃); AuCl(PMe₃); AuCl(Biphenyl(tBu)₂).

3. The process according to claim 2, wherein, the Au(I) complex is AuCl(PPh₃).

4. The process according to claim 1, wherein, the Au(I) complex is used in an amount of 0.05 mol % in homogeneous catalysis.

5. The process according to claim 1, wherein, the process comprises addition of a silver salt to facilitate the [1, 3] O→C rearrangement.

6. The process according to claim 1, wherein, the silver salt is selected from the group consisting of AgSbF₆; AgOAc; AgOTf and AgNTf₂.

7. The process according to claim 1, wherein, the silver salt is AgSbF₆.

8. The process according to claim 1, wherein, the molar ratio of Au [I] complex and additive is in the range of 1:6 to 1:3, more preferably the molar ratio is 1:3.

9. The process according to claim 1, wherein, the process comprises reacting allenyl ethers of Formula 1 with Au(I) complex and silver salt in a molar ratio of 1:3, in presence of aprotic solvents at a temperature of 0° C. to room temperature, to obtain α-substituted acryl aldehydes of Formula 2

10. The process according to claim 1, wherein, the α-substituted acryl aldehydes of Formula 2 comprises
(i) 2-(4-Methoxybenzyl)acrylaldehyde;
(ii) 2-((4-Methoxyphenyl)(phenyl)methyl)acrylaldehyde;
(iii) 3-(4-Methoxyphenyl)-2-methylene-4-phenylbutanal;
(iv) 3-(4-Methoxyphenyl)-2-methyleneheptanal′
(v) 2-(2,4-Dimethoxybenzyl)acrylaldehyde;
(vi) 2-((2,4-Dimethoxyphenyl)(phenyl)methyl)acrylaldehyde;
(vii) 2-(3,4-Dimethoxybenzyl)acrylaldehyde;
(viii) 2-((3,4-Dimethoxyphenyl)(phenyl)methyl)acrylaldehyde;
(ix) 3-(3,4-Dimethoxyphenyl)-2-methylene-4-phenylbutanal;
(x) 2-Methylene-4-phenyl-3-(3,4,5-trimethoxyphenyl)butanal;
(xi) 2-(Phenyl(3,4,5-trimethoxyphenyl)methyl)acrylaldehyde;
(xii) 2-(4-(Dimethylamino)benzyl)acrylaldehyde;
(xiii) 2-((4-(Dimethylamino)phenyl)(phenyl)methyl)acrylaldehyde;
(xiv) 3-(4-(Dimethylamino)phenyl)-2-methyleneheptanal;
(xv) 2-((3,5-Dimethoxyphenyl)(phenyl)methyl)acrylaldehyde;
(xvi) 2-Benzhydrylacrylaldehyde;
(xvii) 2-(6-Methoxy-1,2,3,4-tetrahydronaphthalen-1-yl)acrylaldehyde′
(xviii) 2-((4-Methoxyphenyl)(phenyl)methyl)-3-phenylacrylaldehyde;
(xix) 2-((2,4-Bimethoxyphenyl)(phenyl)methyl)-3-phenylacrylaldehyde;
(xx) Cyclopenta-2,4-dien-1-yl(2-(2-formylallyl)cyclopenta-2,4-dien-1-yl)iron;
(xxi) 3-(1-Methyl-1H-pyrrol-2-yl)-2-methylenebutanal;
(xxii) 2-Methylene-3-(thiophen-2-yl)butanal;
(xxiii) 3-(1-Benzyl-1H-pyrrol-2-yl)-2-methyleneheptanal;
(xxiv) 3-(Benzofuran-2-yl)-2-methylenebutanal;
(xxv) 3-(Benzo[b]thiophen-2-yl)-2-methylenebutanal;
(xxvi) 3-(1-Benzyl-1H-indol-3-yl)-2-methyleneheptanal.

11. A α-substituted acryl aldehydes of Formula 2 comprise
wherein n represents 0,1
“A” is selected from the group consisting of phenyl; benzyl; substituted or unsubstituted furyl, thiophenyl, pyrrolyl, pyridyl, benzofuryl, benzothiophenyl, indolyl, wherein substituents are selected from N—(C₁-C₄)alkyl or, N-arylalkyl;
R is selected from the group consisting of hydrogen, aryl, arylalkyl, (C₁-C₆) linear or branched alkyl;
R¹, R² and R³ are similar or different and independently selected from the group consisting of hydrogen, (C₁-C₄)alkoxy, N—(R⁴)₂, where R⁴ is (C₁-C₆) alkyl;
with a proviso when n is 0, then “A” is selected from the group consisting of benzocyclohexyl and ferrocenyl.
